(19) United States
(12) Patent Application Publication
(10) Pub. No.: US 2025/0049785 A1
(43) Pub. Date: Feb. 13, 2025

(54) METHODS FOR THE TREATMENT OF GLIOBLASTOMA THAT MINIMIZE RADIATION DAMAGE IN SUSCEPTIBLE PATIENTS

(71) Applicant: Rhode Island Hospital, Providence, RI (US)

(72) Inventor: Eric T. Wong, Milton, MA (US)

(21) Appl. No.: 18/724,004

(22) PCT Filed: Dec. 22, 2022
(86) PCT No.: PCT/US2022/082273

§ 371 Date: Jun. 25, 2024

Related U.S. Application Data
(60) Provisional application No. 63/295,306, filed on Dec. 30, 2021.

(51) Int. Cl. A61K 31/495; A61K 31/337; A61K 31/4745; A61N 5/00; A61P 35/00

(52) U.S. Cl. CPC A61K 31/495; A61K 31/337; A61K 31/4745; A61P 35/00; A61N 5/00

(57) ABSTRACT

The embodiments of the present invention provide methods for the treatment of glioblastoma that minimize radiation damage in susceptible patients, such as the elderly. Patients with one or more diagnostic marker of α-synucleinopathy, or other markers of CNS degeneration, receive a modified treatment regimen that minimizes the risk for accelerated encephalopathy or myelopathy from radiation.

### FIELD OF THE INVENTION

The embodiments of the present invention relate to methods for the treatment of glioblastoma that minimize damage in susceptible patients, such as the elderly.

### BACKGROUND OF THE INVENTION

Glioblastoma multiforme (GBM), also known as glioblastoma, recurrent GBM, and/or grade IV astrocytoma, is an extremely aggressive tumor. Symptoms are similar to those of other brain tumors, and may include seizure, nausea and vomiting, headache, memory loss, hemiparesis, and progressive memory, personality, or neurological deficit due to temporal, parietal, occipital, and frontal lobe involvement, as well as subcortical and brainstem involvement.

The standard Stupp protocol has become standard of care for the treatment of GBM since its publication in 2005 and has led to significant survival improvements.¹ It consists of radiotherapy delivered over 6 weeks together with concomitant daily temozolomide (TMZ), followed by adjuvant monthly TMZ. Implementation of the Stupp protocol was reported to result in clinically meaningful and statistically significant increases in survival compared to previous regimens.^{2,3,4}

Unfortunately, elderly patients with GBM have a poor prognosis despite treatment. The standard Stupp protocol, included only patients 70 years of age or younger.⁵ Although a post hoc analysis of the trial cohorts between the age of 65 and 70 did not demonstrate a benefit from the addition of temozolomide,⁶ the combination of hypo-fractionated radiotherapy and temozolomide over 3 weeks still showed improved survival in this population when investigated in a randomized prospective phase 3 trial.⁷ These data suggested that the elderly derives only a small benefit from temozolomide and hypo-fractionated radiotherapy and more investigation is required to determine the optimal balance between efficacy and toxicity in elderly patients. Moreover, older patients are known to be particularly susceptible to both systemic and neurological side effects of chemotherapy and radiation treatments.

Accordingly, there is a need for improved treatment protocols for the treatment of GBM in patients at risk of radiation-induced damage.

### BRIEF SUMMARY OF THE INVENTION

Patients with pre-existing degenerative disease of the brain and spinal cord, such as Parkinson's disease, Lewy body dementia, Alzheimer's disease, fronto-temporal dementia or amyotrophic lateral sclerosis, are at risk for radiation-induced damage. These individuals can be identified by the presence of phosphorylated α-synuclein, tau or β-amyloid in the peripheral nerves, cerebrospinal fluid and/or blood, and/or decreased dopamine uptake in the basal ganglia by 2 standard deviations on DaTscan. Once susceptible patients are identified, alternative treatments can be administered to limit radiation exposure at functional structures of the brain, including the basal ganglia, substantia nigra, frontal lobes, temporal lobes, parietal lobes and occipital lobes, and spare them from delayed radiation-induced damage. These treatment approaches may include (i) ionizing radiation with higher conformality to the tumor, (ii) non-ionizing radiation, (iii) non-ionizing radiation with higher conformality to the tumor, (iv) high energy particle radiation with higher conformality to the tumor, or (v) chemotherapy alone.

The embodiments of the present invention provide improved treatment protocols for the treatment of GBM in patients at risk of radiation-induced damage. The methods of the present invention first require the determination of the presence or absence of one or more diagnostic marker of α-synucleinopathy, or other markers of CNS degeneration, in the patient afflicted with a glioblastoma tumor. Patients with no diagnostic marker of α-synucleinopathy can be treated with radiation and temozolomide according to the standard Stupp protocol. For patients with one or more diagnostic marker of α-synucleinopathy, or other markers of CNS degeneration, however, the treatment protocol is altered to minimize radiation damage.

In one embodiment, susceptible patients with one or more diagnostic marker of α-synucleinopathy, or other markers of CNS degeneration, are treated with drug therapy alone. In an alternative embodiment, susceptible patients are treated with drug therapy in combination with involved-field radiation administered with a more conformal plan to the tumor while avoiding subcortical regions that are at risk of damage. In yet another alternative embodiment, susceptible patients are treated with drug therapy in combination with high energy particle radiation administered with a more conformal plan to the tumor while avoiding subcortical regions that are at risk of damage. In yet another alternative embodiment, susceptible patients are treated with drug therapy in combination with ionizing radiation administered with a more conformal plan to the tumor while avoiding subcortical regions that are at risk of damage. In yet another alternative embodiment, susceptible patients are treated with drug therapy in combination with non-ionizing radiation. In yet another alternative embodiment, susceptible patients are treated with non-ionizing radiation alone.

In some embodiments, the drug therapy can be a chemotherapeutic agent, an immunotherapeutic agent, an antibody-drug conjugate, an antiangiogenic agent, a neuroprotection agent, a senolytic agent, an anti-inflammatory agent, or a combination thereof. In some embodiments, the chemotherapeutic agent can be an alkylating agent, an anthracycline, a cytoskeletal disruptor, an epothilone, a histone deacetylase inhibitor, an inhibitor of topoisomerase I, an inhibitor of topoisomerase II, a kinase inhibitor, a nucleotide analog, a peptide antibiotic, a platinum-based agent, a retinoid, a vinca alkaloid, or a combination thereof. In some embodiments, the alkylating agent can be temozolomide, irinotecan and paclitaxel, or a combination thereof.

Diagnostic markers of α-synucleinopathy include: (i) phosphorylated α-synuclein; (ii) Lewy body or bodies, and/or (iii) decreased dopamine levels in the stratum of the subject or other markers of CNS degeneration include (i) phosphorylated tau; (ii) β-amyloid; (iii) transactive response DNA binding protein 43 kDa (TDP-43); (iv) fused in sarcoma (FUS); and (v) telomeric repeat-binding factor 2 (Trf2). Phosphorylated α-synuclein can be identified in peripheral nerve endings from a skin punch biopsy or as α-synuclein aggregates in a sample of brain and/or spinal cord tissue, cerebrospinal fluid, or blood obtained from the subject. The decreased dopamine levels in the stratum of the subject can be measured by DaTscan. Other markers of CNS degeneration can be identified directly or indirectly in brain or spinal cord tissue, cerebrospinal fluid, or blood.

In an alternative embodiment, one or more of the following α-synucleinopathy diagnostic markers, or other markers of CNS degeneration, are identified in a sample of cerebrospinal fluid or blood obtained from the subject: phosphorylated α-synuclein; phosphorylated tau; β-amyloid; TDP-43; FUS; and/or Trf2.

Other implementations are also described and recited herein.

### BRIEF DESCRIPTION OF THE DRAWINGS

For the purpose of illustration, certain embodiments of the present invention are shown in the drawings described below. It should be understood, however, that the invention is not limited to the precise arrangements, dimensions, and instruments shown. In the drawings:

FIG. 1A-D shows a non-contrast head computerized tomography (CT) scan of a patient with an isocitrate dehydrogenase 1 (IDH-1) wild-type glioblastoma before and after surgery, radiation and chemotherapy. MRI from post-gadolinium T1-weighted (FIG. 1A) and FLAIR sequences (FIG. 1B) demonstrating irregular cystic enhancement and adjacent cerebral edema. The surgical cavity after gross total resection and external beam involved-field radiation as seen on the post-gadolinium T1-weighted (FIG. 1C) and FLAIR sequences (FIG. 1D).

FIG. 2 depicts radiation mapping diagrams. The clinical tumor volume, which consists of the surgical cavity after gross total resection, FLAIR positive region and a margin of 2 cm, received 4600 cGy in 200 cGy per fraction over 23 days (FIG. 2A). This was followed by a boost to the surgical cavity and FLAIR positive region to 1400 cGy also in 200 cGy per fraction for an additional 7 days (FIG. 2B).

FIG. 3 shows the brain autopsy results findings in the brainstem, which showed a slight loss of myelination due to the radiation (FIG. 3A). There is positive immunohistochemical staining for neuronal cytoplasmic α-synuclein (FIG. 3B, arrow) and Lewy body (FIG. 3C, arrow).

FIG. 4 shows hematoxylin and eosin staining of the left temporal pole. Expected inflammatory lymphocytes were seen in the brain parenchyma (FIG. 4A). The Ki-67 proliferation index was low (FIG. 4B) and there were observable pyknotic tumor nuclei (FIG. 4C).

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention. It is to be appreciated that certain aspects, modes, embodiments, variations and features of the invention are described below in various levels of detail in order to provide a substantial understanding of the present invention.

### Abbreviations

The following abbreviations are used in the present description: 53BP1, p53 binding protein 1; ATM, ataxia telangiectasia mutated; ATRX, α-thalassemia mental retardation syndrome; cerebrospinal fluid; cGy, centigray; CNS, central nervous system; CT, computed tomography; DaTscan, dopamine active transfer scan; FUS, fused in sarcoma; γH2Ax, gamma H2A histone family member X; GFAP, glial fibrillary acidic protein; HSV, herpes simplex virus; IDH, isocitrate dehydrogenase; Ki-67, marker of proliferation 67; MRI, magnetic resonance imaging; Olig-2, oligodendrocyte transcription factor-2; PARP, poly-ADP-ribose polymerase; PCR, polymerase chain reaction; TDP-43, transactive response DNA binding protein 43 kDa; Trf2, telomeric repeat-binding factor 2; VZV, varicella-zoster virus.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex.

As used herein, the terms “effective amount” and “therapeutically-effective amount” include an amount sufficient to prevent or ameliorate a manifestation of disease or medical condition, such as alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

As used herein, the term “long-term” administration means that (i) the therapeutic agent or drug is administered for a period of at least 12 weeks, and/or (ii) the therapeutic agent or radiation is administered for a period of at least 1 day. This includes that the therapeutic agent or drug is administered such that it is effective over, or for, a period of at least 12 weeks and does not necessarily imply that the administration itself takes place for 12 weeks, e.g., if sustained release compositions or long acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more. This also includes that the therapeutic agent or radiation is administered for a period of at least 1 day. This includes that the therapeutic agent or radiation is administered such that it is effective over, or for, a period of at least 1 day.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

### Cancer-Related Definitions

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. CNS cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

A subject that has a cancer or a tumor is a subject having objectively measurable cancer cells present in the subject's body. Included in this definition are malignant, actively proliferative cancers, as well as potentially dormant tumors or micrometastatses. Cancers which migrate from their original location and seed other vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

A subject can be one who has been previously diagnosed with or identified as suffering from or having a condition in need of treatment (e.g., a cancer) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition or one or more complications related to a condition or a subject who does not exhibit risk factors. A “subject in need” of treatment for a particular condition can be a subject having that condition, diagnosed as having that condition, or at risk of developing that condition.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragées, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragées, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically-acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher et al. (1996).⁸

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;⁹ The Encyclopedia of Molecular Cell Biology and Molecular Medicine;¹⁰ Molecular Biology and Biotechnology: a Comprehensive Desk Reference;¹¹ Immunology;¹² Janeway's Immunobiology;¹³ Lewin's Genes XI;¹⁴ Molecular Cloning: A Laboratory Manual;¹⁵ Basic Methods in Molecular Biology;¹⁶ Laboratory Methods in Enzymology;¹⁷ Current Protocols in Molecular Biology (CPMB);¹⁸ Current Protocols in Protein Science (CPPS);¹⁹ and Current Protocols in Immunology (CPI).²⁰

### Radiation Technologies

Radiation pertains to energy derived from any frequency of the electromagnetic spectrum or high energy particles, including but not limited to, (i) ionizing portion of the electromagnetic spectrum, (ii) non-ionizing portion of the electromagnetic spectrum, or (iii) high energy particle radiotherapy including but not limited to proton beam radiotherapy or boron-neutron capture radiotherapy. Technology or technologies are means of delivering the energy to a human or mammal for the treatment of cancer.

Ionizing radiation consists of high energy waves that can eject electrons on its path causing damage to biological processes or entities, including but not limited to cancer cells, tumor, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

Non-ionizing radiation consists of lower energy waves that does not eject electrons but still causing damage to biological processes or entities, including but not limited to cancer cells, tumor, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

High energy particle radiotherapy consists of particles accelerated to a high energy state causing damage to biological processes or entities, including but not limited to cancer cells, tumor, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

Conformality refers to a method of image-guided delivery of (i) ionizing radiation, (ii) non-ionizing radiation, or (iii) high energy particle radiotherapy, to a target, including but not limited to, cancer cells, tumor, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome, while limiting the delivery of ionizing or non-ionizing radiation to normal tissue in a human or mammal.

A combination of ionizing radiation and conformality can increase the therapeutic window to the target while minimizing side effects to the surrounding normal tissue. An example of ionizing radiation include, but not limited to, radiation from a linear accelerator or Linac radiation, stereotactic radiosurgery such as Gamma Knife or CyberKnife.

A combination of non-ionizing radiation and conformality can increase the therapeutic window to the target while minimizing side effects to the surrounding normal tissue. An example of non-ionizing radiation include, but not limited to, (i) infrared or thermal therapy or (ii) tumor treating electric fields (also called Tumor Treating Fields).

A combination of high energy particle radiotherapy and conformality can increase the therapeutic window to the target while minimizing side effects to the surrounding normal tissue. An example of high energy particle radiotherapy include, but not limited to, (i) proton beam radiotherapy or (ii) boron neutron capture therapy.

A combination of ionizing radiation, conformality, and drug can increase the therapeutic window to the target while minimizing side effects to the surrounding normal tissue. An example of ionizing radiation includes, but is not limited to, (i) radiation from a linear accelerator or Linac radiation, or (ii) stereotactic radiosurgery such as Gamma Knife or CyberKnife.

A combination of non-ionizing radiation, conformality and drug can increase the therapeutic window to the target while minimizing side effects to the surrounding normal tissue. An example of non-ionizing radiation includes, but is not limited to, (i) infrared or thermal therapy or (ii) tumor treating electric fields (also called Tumor Treating Fields).

A combination of high energy particle radiotherapy, conformality and drug can increase the therapeutic window to the target while minimizing side effects to the surrounding normal tissue. An example of high energy particle radiotherapy includes, but is not limited to, (i) proton beam radiotherapy or (ii) boron neutron capture therapy.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

Treatment Complications in Elderly Patients with Glioblastoma

The Example section of the present disclosure describes an elderly man who underwent a complete resection of an IDH-1 wild-type glioblastoma. He experienced rapid neurocognitive decline after concomitant temozolomide and external beam involved-field radiotherapy to the left temporal lobe resection cavity, consisting of progressive confusion, word-finding difficulty, and imbalance. Radiation at a dose of 6,000 cGy was delivered to the ipsilateral putamen and globus pallidus, 3,000 cGy to the brainstem and up to 1,800 cGy to the contralateral putamen and globus pallidus.

He died suddenly four months from his initial diagnosis. Post-mortem examination revealed atherosclerosis of multiple coronary arteries with significant focal narrowing of the right coronary arteries with a proximal stent. His lungs had emphysematous changes but there was no evidence of pulmonary thromboembolism. But examination of his brain revealed diffuse presence of neuronal cytoplasmic Lewy bodies that are immunohistochemically stained positive for α-synuclein in the midbrain and pons, as well as bilateral amygdala, putamen and globus pallidus. Expected findings include inflammatory lymphocytes and pyknotic tumor nuclei. But no amyloid plaques and only rare neurofibrillary tangles were noted in the cortex adjacent to the hippocampi.

This case raised the possibility that radiation and temozolomide may accelerate the clinical deterioration of glioblastoma patients with concurrent diffuse Lewy body disease. Therefore, it was concluded that α-synucleinopathy in the brain may be a negative modifier of clinical outcome in this population.

Predisposition for Treatment Complications in Elderly Patients with Glioblastoma

Population studies have shown that over 60% of the individuals 65 years old or older have two or more systemic co-morbidities, including concurrent heart disease, chronic obstructive pulmonary disease, diabetes, kidney disease and stroke.²¹ Furthermore, the prevalence of dementia also increases in these same patients. Alzheimer's disease and vascular dementia represents the most common and second most common type of memory disorders, respectively, occurring at a combined rate of 5% in the seventh, 25% in the eighth and 37% in the ninth decade.²² Lewy body dementia is third most common and comprises 5% of all dementia cases.^{23,24}

Lewy bodies are found in patients with dementia with Lewy bodies, Parkinson's disease and multisystem atrophy.²⁵ They are intra-neuronal aggregates of α-synuclein fibrils phosphorylated at the serine 129 residues.^{26,27} Misfolded or overexpressed cytoplasmic α-synucleins can damage mitochondria and make neurons susceptible to oxidative stress.^{28,29} It is notable that ionizing radiation can also cause persistent oxidative stress within the irradiated tissue by damaging the mitochondria and generating reactive oxygen species, in the form of oxygen and hydroxyl radicals as well as hydrogen peroxide.^{30,31,32}

Elderly patients with glioblastoma have a shortened survival. They also suffer from treatment complications including myelosuppression, steroid side effects as well as cognitive impairment from radiation, chemotherapy or both.

These elderly patients often develop neurocognitive dysfunction after radiotherapy. Delayed encephalopathy or dementia is a dreaded irreversible complication of whole brain radiotherapy. This is thought to be a result of hippocampal damage, a site of continued neural genesis during adulthood. Indeed, bilateral hippocampal sparing whole brain radiation with memantine preserves neurocognitive function better than the traditional fractionated whole brain radiotherapy.³³ Still, there is heterogeneity in the outcome of these patients and sparing of the hippocampi may not be enough to protect elderly patients. This is because the majority of neurodegenerative diseases have misfolded proteins that are localized in the nucleus and play a role in neuronal DNA repair in regions of the brain other than the hippocampi, including tau in Alzheimer's disease, TDP-43 and FUS in frontotemporal lobar dementia and amyotrophic lateral sclerosis,^{34,35} Trf2 in neurodegeneration from welding fume exposure^{36,37} and α-synucleinopathies in diffuse Lewy body disease, Parkinson's disease and multisystem atrophy.³⁸ Indeed, our patient's autopsy revealed no amyloid plaques and only rare neurofibrillary tangles in the cortex adjacent to the hippocampi. Furthermore, α-synuclein and tau are known to spread in the CNS from one nerve cell to another in a prion-like fashion.³⁹ Therefore, focal and whole brain radiotherapy or spinal irradiation can accelerate neurodegeneration in the brain or spinal cord resulting in progressive delayed radiation-induced encephalopathy or myelopathy.

Minimizing Treatment Complications in Susceptible Patients with Glioblastoma

α-Synuclein is localized in the pre-synaptic region and its major function is to facilitate neuronal signaling.⁴⁰ This protein is also found in the nucleus. In animal models, nuclear α-synuclein co-localizes with DNA damage response proteins ATM, γH2Ax and 53BP1, which are important for the repair of single- and double-strand breaks.⁴¹ It has been shown that α-synuclein binds to double-stranded DNA damaged by radiomimetic bleomycin and it may play a role with the Ku70/Ku80 complex in non-homologous end-joining repair.^{42,43} Furthermore, upon single-strand DNA damage, there is a 25-fold increase in the development of neurotoxic aggregates of α-synuclein and poly-ADP-ribose, the latter of which is generated by activated poly-ADP-ribose polymerase (PARP).⁴⁴ Importantly, high levels of Lewy bodies are often found in the amygdala and the extent of pathology in patients with Lewy body disease correlates with the level of DNA double-strand breaks.⁴⁵ Since radiation triggers double-strand DNA breaks and temozolomide causes N-alkylation of purines requiring base-excision repair by PARP,⁴⁶ glioblastoma patients with a high burden of α-synucleopathy or Lewy bodies may be particularly susceptible to neuronal damage induced by concomitant radiation and temozolomide.

The elderly patient described in the Example section of the present disclosure underwent involved-field radiotherapy for his left temporal glioblastoma and the irradiated hippocampus, putamen and globus pallidus on the left received up to 6,000 cGy of radiation while the right encountered a significantly lesser amount at 1,800 cGy or lower. Still, the patient experienced a rapid decline in his neurocognitive function immediately after radiation and post-mortem examination revealed Lewy bodies in the subcortical regions of his brain. Therefore, this suggests that the patient already had subclinical Lewy body disease while undergoing external beam radiotherapy, which might have potentiated α-synuclein-induced oxidative stress in neurons and accelerated his neurological decline.

There are currently diagnostic markers of α-synucleinopathy for patients with subclinical disease. Phosphorylated α-synuclein can be identified in peripheral nerve endings from skin punch biopsy and decreased dopamine levels can be observed in the stratum by DaTscan.^{47,48} Furthermore, certain forms of α-synuclein aggregates can be identified in the cerebrospinal fluid.⁴⁹

Therefore, according to the present invention, elderly patients with glioblastoma would benefit from these diagnostic studies and, when α-synucleinopathy is diagnosed, involved-field radiation can be administered with a more conformal plan to the tumor while avoiding subcortical regions that are at risk of damage. Alternatively, elderly glioblastoma patients could forgo radiation and be treated with temozolomide alone.⁵⁰ Similarly, for those at risk for Alzheimer's disease, phosphorylated tau and β-amyloid can be detected in the cerebrospinal fluid,⁵¹ and patients with high levels of these misfolded proteins would benefit from an alternative treatment plan. Similarly, diagnostic detection for TDP-43,⁵² FUS, or Trf2 in the clinic would also be helpful in minimizing radiation damage in susceptible patients.

According to the present invention, the detection of any of the above phosphorylated or misfolded proteins, either individually or in combination, will help identify patients at risk for accelerated encephalopathy or myelopathy from radiation and alter the treatment approaches to minimize the risks for these susceptible patients.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Example 1 Case Presentation

### Diagnosis

A 77-year-old right-handed Caucasian man had developed confusion, word-finding difficulty and slurred speech. These symptoms included making phone calls to his wife and asking her non-sensible questions. He lost his words intermittently and forgot names of his family members on a number of occasions. He recounted old stories as if he were perseverating and answered questions in the affirmative without actually hearing the question. His mood also became more irritable. His past medical history was notable for a skull fracture after fall from horseback riding when he was a teenager. At age 75, the patient had undergone a neurocognitive evaluation from a memory disorder clinic in a major academic medical center and he had been diagnosed with a non-specific memory disorder.

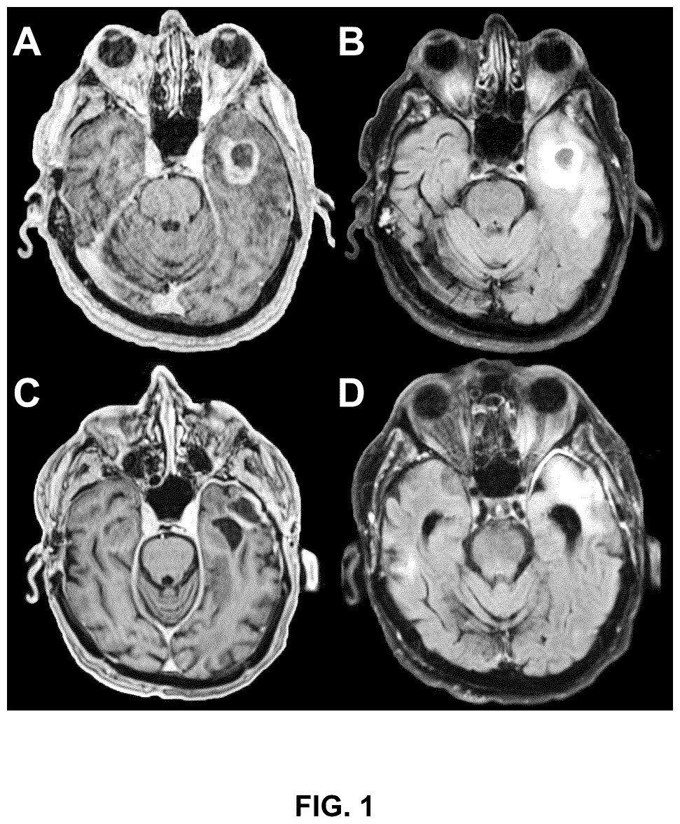

A non-contrast head CT, obtained after 3 months into his progressive symptoms, showed a hypodense mass in the left temporal lobe of the brain. On the following day, a CT of the torso was unremarkable, but a gadolinium-enhanced head MRI revealed a cystic enhancing mass with central necrosis, measuring 2.5×2.4×2.7 cm, and associated with extensive T2 and FLAIR signals extending from the inferior left temporal lobe to the insular cortex as well as the internal capsule and the peri-ventricular white matter near the occipital horn on the left side (see FIG. 1). There was a 6 mm rightward midline shift together with mild effacement of the ambient cistern.

### Treatment

The patient underwent a gross total resection of the mass 4 days after the MRI. Pathology showed IDH-1 wild-type glioblastoma and the histological features are notable for dense cellularity, moderate to severe atypia and easily identified mitoses. Immunohistochemical staining was negative for IDH-1 R132H mutation but positive for ATRX, GFAP, Olig-2 and p53. The Ki-67 index showed 20-25% positivity indicating a moderate proliferation rate.

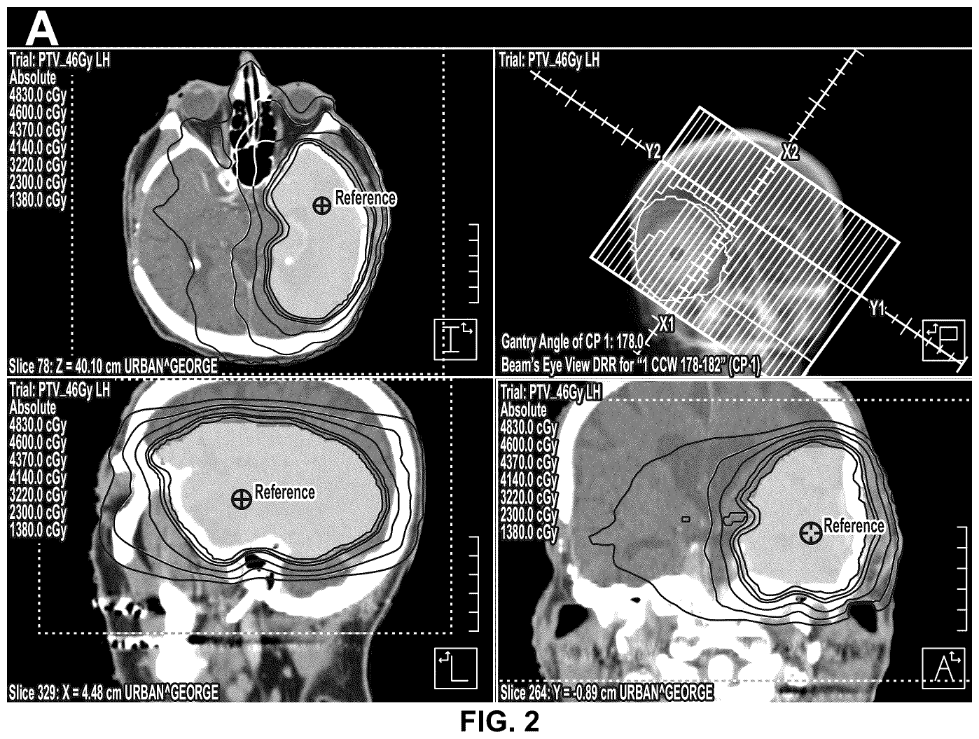

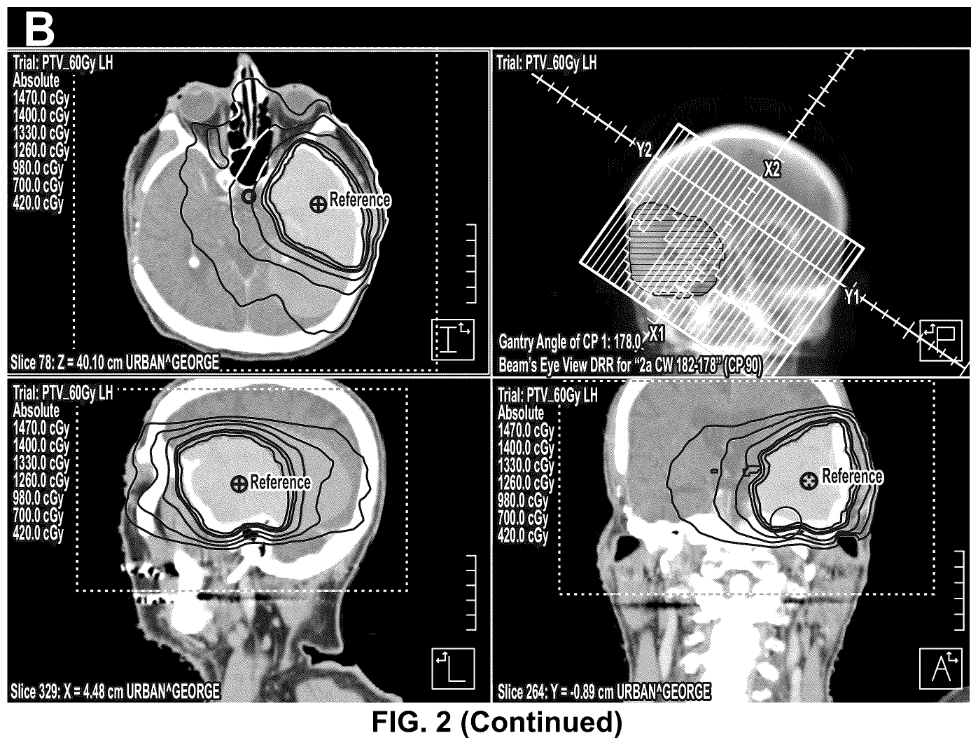

Three weeks after surgery, the patient started involved-field radiotherapy and concomitant daily TMZ, but only two weeks of chemotherapy was administered due to low blood counts, and six weeks of radiation treatment was eventually completed two months after the diagnosis. The radiation administered consisted of standard external beam radiotherapy. The left temporal postsurgical cavity as well as the ipsilateral putamen and globus pallidus were included in the maximum 6,000 cGy isodose line (see FIG. 2). The brainstem was covered in the 3,000 cGy isodose line while the contralateral putamen and globus pallidus received up to 1,800 cGy of radiation.

### Outcome

The patient's cognitive status rapidly deteriorated rapidly after radiation and TMZ. He became more forgetful and developed imbalance. He required 24-hour supervision for his daily activities. A lumbar puncture was performed 4 months after glioblastoma diagnosis and showed 1 white blood cell, 1 red blood cell, 50 protein, 66 glucose, 18 lactate dehydrogenase, negative VDRL, no detectable immunoglobulin on immunofixation, negative PCR for HSV-1, HSV-2 and VZV, and negative cytology for malignant cells. Phospho-tau was not elevated at 29.6 pg/ml (normal <54 mg/ml). He died unexpectedly from sudden death 2 months after completion of radiation or 4 months after establishment of the glioblastoma diagnosis.

### Autopsy

Autopsy of the body showed atherosclerosis of multiple coronary arteries, including (i) 25-50% narrowing of left main artery, (ii) 25-50% narrowing of the left anterior descending artery, and (iii) focal 75% narrowing of the right coronary arteries with a proximal stent. His lungs had emphysematous changes but there was no evidence of pulmonary thromboembolism.

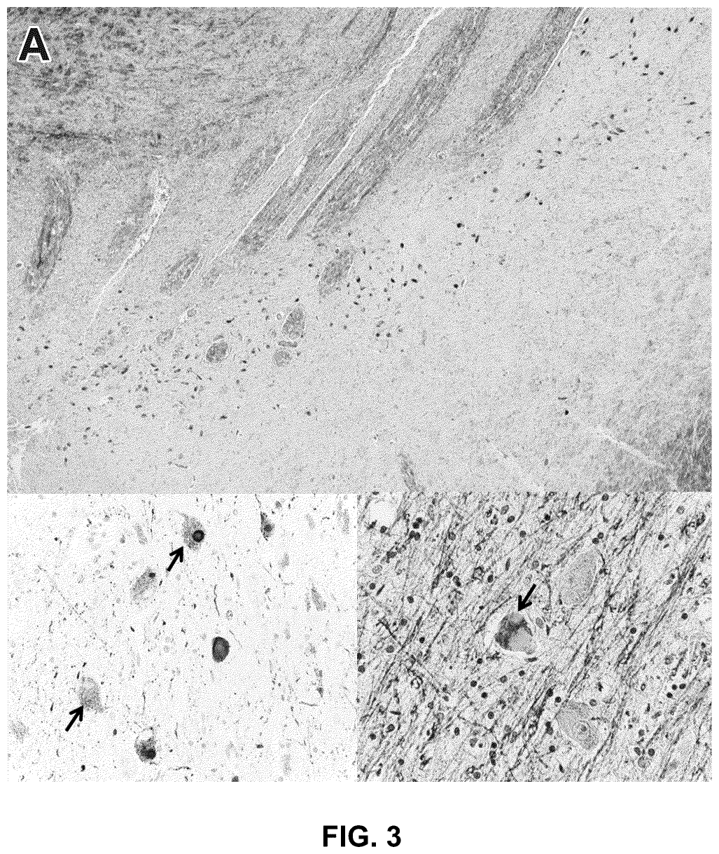

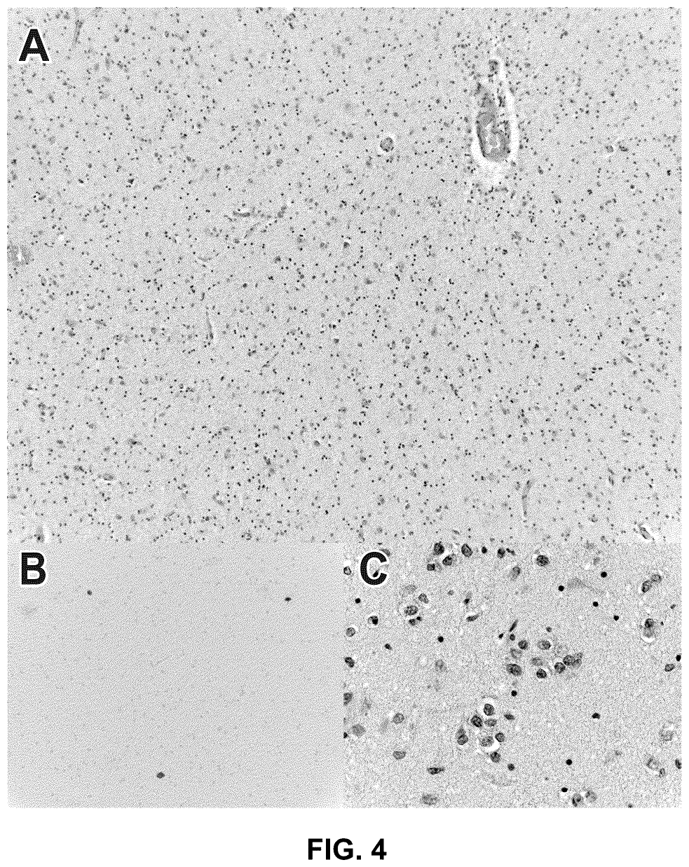

The patient's brain autopsy revealed both neuronal cytoplasmic inclusions and Lewy bodies that were immunohistochemically stained positive for α-synuclein in the midbrain (see FIG. 3), pons, amygdala, putamen and globus pallidus. There were inflammatory small lymphocytes and tumors cells with pyknotic nuclei (see FIG. 4), but no amyloid plaques and only rare neurofibrillary tangles in the cortex adjacent to the hippocampi.

### Discussion and Conclusions

The patient's rapid neurocognitive decline after radiation suggests that he had an intrinsic vulnerability to radiotherapy. The differential diagnosis of this rapid decline included communicating hydrocephalus, subacute encephalopathy or subclinical neurodegenerative disease of the brain that was accelerated by radiation. A lumbar puncture showed normal CSF pressure and his neurocognitive dysfunction did not improve after the procedure. Furthermore, phospho-tau level was low in the cerebrospinal fluid indicating either no tauopathy or at least a low burden of disease in the brain. Although the exact pathophysiology of subacute radiation-induced encephalopathy is unknown, this condition is usually reversible when given enough time and corticosteroid usually accelerates patient recovery. However, this patient did not show neurocognitive improvement despite the addition of dexamethasone. Therefore, a post-mortem examination of his brain was performed, which revealed a widespread distribution of Lewy bodies in the subcortical regions of his brain, indicating a pathological diagnosis of diffuse Lewy body disease.

Lewy bodies are found in patients with dementia with Lewy bodies, Parkinson's disease and multisystem atrophy.⁵³ They are intra-neuronal aggregates of α-synuclein fibrils phosphorylated at the serine 129 residues.^{54,55} Misfolded or overexpressed cytoplasmic α-synucleins can damage mitochondria and make neurons susceptible to oxidative stress.^{56,57} It is notable that ionizing radiation can also cause persistent oxidative stress within the irradiated tissue by damaging the mitochondria and generating reactive oxygen species, in the form of oxygen and hydroxyl radicals as well as hydrogen peroxide.^{58,59,60}

The patient underwent involved-field radiotherapy for his left temporal glioblastoma and the irradiated hippocampus, putamen and globus pallidus on the left received up to 6,000 cGy of radiation while the right encountered a significantly lesser amount at 1,800 cGy or lower. Still, the patient experienced a rapid decline in his neurocognitive function immediately after radiation and post-mortem examination revealed Lewy bodies in the subcortical regions of his brain. Therefore, this suggests that the patient already had subclinical Lewy body disease while undergoing external beam radiotherapy, which might have potentiated α-synuclein-induced oxidative stress in neurons and accelerated his neurological decline.

Elderly individuals often develop neurocognitive dysfunction after radiotherapy. Delayed encephalopathy or dementia is a dreaded irreversible complication of whole brain radiotherapy. This is thought to be a result of hippocampal damage, a site of continued neural genesis during adulthood. Indeed, bilateral hippocampal sparing whole brain radiation with memantine preserves neurocognitive function better than the traditional fractionated whole brain radiotherapy.⁶¹ Still, there is heterogeneity in the outcome of these patients and sparing of the hippocampi may not be enough to protect elderly patients. This is because the majority of neurodegenerative diseases have misfolded proteins that are localized in the nucleus and play a role in neuronal DNA repair in regions of the brain other than the hippocampi, including tau in Alzheimer's disease, TDP-43 and FUS in frontotemporal lobar dementia and amyotrophic lateral sclerosis,^{62,63} Trf2 in neurodegeneration from welding fume exposure^{64,65} and α-synucleinopathies in diffuse Lewy body disease, Parkinson's disease and multisystem atrophy.⁶⁶ Indeed, our patient's autopsy revealed no amyloid plaques and only rare neurofibrillary tangles in the cortex adjacent to the hippocampi. Furthermore, α-synuclein and tau are known to spread in the CNS from one nerve cell to another in a prion-like fashion.⁶⁷ Therefore, focal and whole brain radiotherapy or spinal irradiation appear to accelerate neurodegeneration in the brain or spinal cord resulting in progressive delayed radiation-induced encephalopathy or myelopathy.

As described above, there are currently diagnostic markers of α-synucleinopathy for patients with subclinical disease. Phosphorylated α-synuclein can be identified in peripheral nerve endings from skin punch biopsy and decreased dopamine levels can be observed in the stratum by DaTscan.^{68,69} Furthermore, certain forms of α-synuclein aggregates can be identified in the cerebrospinal fluid.⁷⁰ Similarly, for those at risk for Alzheimer's disease, phosphorylated tau and β-amyloid can be detected in the cerebrospinal fluid⁷¹ and patients with high levels of these misfolded proteins may need an alternative treatment plan. Similarly, diagnostic detection for TAR DNA binding protein 43 (TDP-43),⁷² Fused in Sarcoma (FUS), or telomeric repeat-binding factor 2 (Trf2) in the clinic can also be helpful.

Therefore, according to the present disclosure, elderly patients with glioblastoma would benefit from these diagnostic studies and, when α-synucleinopathy or other markers of CNS degeneration is diagnosed, involved-field radiation can be administered with a more conformal plan to the tumor while avoiding subcortical regions that are at risk of damage. Alternatively, elderly glioblastoma patients can forgo radiation and be treated with temozolomide alone.⁷³ According to the present invention, the use of any of the above phosphorylated or misfolded proteins, either individually or in combination, can help identify patients at risk for accelerated encephalopathy or myelopathy from radiation, and the treatment approaches can be altered to minimize the risks for these susceptible patients.

### REFERENCES

¹ Stupp, R., et al. (2005). “Radiotherapy plus Concomitant and Adjuvant Temozolomide for Glioblastoma.” N. Engl. J. Med. 352: 987-996.
² Stu pp, R., et al. (2009). “Effects of radiotherapy with concomitant and adjuvant temozolomide versus radiotherapy alone on survival in glioblastoma in a randomised phase III study: 5-year analysis of the EORTC-NCIC trial.” Lancet Oncol. 10(5): 459-466.
³ Stu pp, R., et al. (2017). “Prospective, multi-center phase III trial of tumor treating fields together with temozolomide compared to temozolomide alone in newly diagnosed glioblastoma.” Presented at: 2017 ANNUAL MEETING OF THE AMERICAN ASSOCIATION FOR CANCER RESEARCH; Washington, DC. Oral presentation LBA AACR CT007.
⁴ Toms, S. A., et al. (2019). “Increased compliance with tumor treating fields therapy is prognostic for improved survival in the treatment of glioblastoma: a subgroup analysis of the EF-14 phase III trial.” J. Neurooncol. 141(2): 467-473.
⁵ Stu pp, R., et al. (2009). “Effects of radiotherapy with concomitant and adjuvant temozolomide versus radiotherapy alone on survival in glioblastoma in a randomised phase III study: 5-year analysis of the EORTC-NCIC trial.” Lancet Oncol. 10(5): 459-466.
⁶ Laperriere N, Weller M, Stupp R, Perry J R, Brandes A A, Wick W, et al. Optimal management of elderly patients with glioblastoma. Cancer Treat Rev. 2013; 39(4): p. 350-357.
⁷ Perry, J. R., et al. (2017). “Short-course radiation plus temozolomide in elderly patients with glioblastoma.” N. Engl. J. Med. 376(11): 1027-1037.
⁸ Isselbacher, et al. (1996). HARRISON'S PRINCIPLES OF INTERNAL MEDICINE, 13 ed., 1814-1882.
⁹ THE MERCK MANUAL OF DIAGNOSIS AND THERAPY, (2011). 19^{th} Edition, published by Merck Sharp & Dohme Corp., (ISBN 978-0-911910-19-3).
¹⁰ THE ENCYCLOPEDIA OF MOLECULAR CELL BIOLOGY AND MOLECULAR MEDICINE, Robert S. Porter et al. (eds.), published by Blackwell Science Ltd., 1999-2012 (ISBN 9783527600908).
¹¹ MOLECULAR BIOLOGY AND BIOTECHNOLOGY: A COMPREHENSIVE DESK REFERENCE, (1995). Robert A. Meyers (ed.), published by VCH Publishers, Inc. (ISBN 1-56081-569-8).
¹² IMMUNOLOGY, (2006). Werner Luttmann, published by Elsevier.
¹³ JANEWAY'S IMMUNOBIOLOGY, (2014). Kenneth Murphy, Allan Mowat, Casey Weaver (eds.), Taylor & Francis Limited, (ISBN 0815345305, 9780815345305).
¹⁴ LEWIN'S GENES XI, (2014). published by Jones & Bartlett Publishers (ISBN-1449659055).
¹⁵ Michael Richard Green and Joseph Sambrook, (2012). MOLECULAR CLONING: A LABORATORY MANUAL, 4th ed., Cold Spring Harbor Laboratory Press, Cold Spring Harbor, N.Y., USA (ISBN 1936113414).
¹⁶ Davis et al., (2012). BASIC METHODS IN MOLECULAR BIOLOGY, Elsevier Science Publishing, Inc., New York, USA (ISBN 044460149X).
¹⁷ LABORATORY METHODS IN ENZYMOLOGY: DNA, (2013). Jon Lorsch (ed.) Elsevier (ISBN 0124199542).
¹⁸ CURRENT PROTOCOLS IN MOLECULAR BIOLOGY (CPMB), (2014). Frederick M. Ausubel (ed.), John Wiley and Sons (ISBN 047150338X, 9780471503385).
¹⁹ CURRENT PROTOCOLS IN PROTEIN SCIENCE (CPPS), (2005). John E. Coligan (ed.), John Wiley and Sons, Inc.
²⁰ CURRENT PROTOCOLS IN IMMUNOLOGY (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
²¹ Boersma, P., et al. (2020). “Prevalence of multiple chronic conditions among US adults, 2018.” Prev. Chronic. Dis. 17: 200130.
²² Plassman, B. L., et al. (2007). “Prevalence of dementia in the United States: the aging, demographics, and memory study.” Neuroepidemiology 29(1-2): 125-132.
²³ Kane, J. P., et al. (2018). “Clinical prevalende of Lewy body dementia.” Alzheimers Res. Ther. 10(1): 19.
²⁴ Hogan, D. B., et al. (2016). “The prevalence and incidence of dementia with Lewy bodies: a systematic review.” Can. J. Neurol. Sci. 43(Suppl 1): S83-S95.
²⁵ Bendor, J. T., et al. (2013). “The function of α-synuclein.” Neuron 79(6): 1044-1066.
²⁶ Spillantini, M. G., et al. (1997). “α-Synuclein in Lewy bodies.” Nature 388(6645): 839-840.
²⁷ Fujiwara, H., et al. (2002). “α-Synuclein is phosphorylated in synucleinopathy lesions.” Nat. Cell Biol. 2002; 4(2): 160-164.
²⁸ Milanese, C., et al. (2018). “Activation of the DNA damage response in vivo in synocleinopathy models of Parkinson's disease.” Cell Death Dis. 9(8): 818.
²⁹ Schaser, A. J., et al. (2019). “Alpha-synuclein is a DNA binding protein that modulates DNA repair with implications for Lewy body disorders.” Sci. Rep. 9(1): 10919.
³⁰ Goldstein, M. and Kastan, M. B. (2015). “The DNA damage response: implications for tumor responses to radiation and chemotherapy.” Annu. Rev. Med. 66: 129-143.
³¹ Kam, T., et al. (2018). “Poly(ADP-ribose) drives pathologic α-synuclein neurodegeneration in Parkinson's disease.” Science 362(6414): eaat8407.
³² Brown, P. D., et al. (2020). “Hippocampal avoidance during whole-brain radiotherapy plus memantine for patients with brain metastases: phase III trial NRG Oncology CC001.” J. Clin Oncol. 38(10): 1019-1029.
³³ Brown, P. D., et al. (2020). “Hippocampal avoidance during whole-brain radiotherapy plus memantine for patients with brain metastases: phase III trial NRG Oncology CC001.” J. Clin Oncol. 38(10): 1019-1029.
³⁴ Mitra, J. and Hegde, M. L. (2019). “A commentary on TDP-43 and DNA damage response in amyotrophic lateral sclerosis.” J. Exp. Neurosci. 13: 1179069519880166.
³⁵ Dormann, D. and Haass, C. (2013). “Fused in sarcoma (FUS): an oncogene goes awry in neurodegeneration.” Mol. Cell Neurosci. 56: 475-486.
³⁶ Shoeb, M., et al. (2020). “A possible relationship between telomere length and markers of neurodegeneration in rate brain after welding fume inhalation exposure.” Environ. Res. 180: 108900.
³⁷ Pereira C D, et al. (2020). “Nuclear accumulation of LAP1:TRF2 complex during DNA damage response uncovers a novel role for LAP1.” Cells 9(8): 1804.
³⁸ Bendor, J. T., et al. (2013). “The function of α-synuclein.” Neuron 79(6): 1044-1066.
³⁹ Vasili, E., et al. (2019). “Spreading of α-synuclein and tau: a systematic comparison of the mechanisms involved.” Front. Mol. Neurosci. 12: 107.
⁴⁰ Bendor, J. T., et al. (2013). “The function of α-synuclein.” Neuron 79(6): 1044-1066.
⁴¹ Milanese, C., et al. (2018). “Activation of the DNA damage response in vivo in synocleinopathy models of Parkinson's disease.” Cell Death Dis. 9(8): 818.
⁴² Schaser, A. J., et al. (2019). “Alpha-synuclein is a DNA binding protein that modulates DNA repair with implications for Lewy body disorders.” Sci. Rep. 9(1): 10919.
⁴³ Goldstein, M. and Kastan, M. B. (2015). “The DNA damage response: implications for tumor responses to radiation and chemotherapy.” Annu. Rev. Med. 66: 129-143.
⁴⁴ Kam, T., et al. (2018). “Poly(ADP-ribose) drives pathologic α-synuclein neurodegeneration in Parkinson's disease.” Science 362(6414): eaat8407.
⁴⁵ Schaser, A. J., et al. (2019). “Alpha-synuclein is a DNA binding protein that modulates DNA repair with implications for Lewy body disorders.” Sci. Rep. 9(1): 10919.
⁴⁶ Goldstein, M. and Kastan, M. B. (2015). “The DNA damage response: implications for tumor responses to radiation and chemotherapy.” Annu. Rev. Med. 66: 129-143.
⁴⁷ Gibbons, C. H., et al. (2016). “The diagnostic discrimination of cutaneous α-synuclein deposition in Parkinson's disease.” Neurology 87(5): 505-512.
⁴⁸ Isaacson, S. H., et al. (2017). “Clinical utility of DaTscan imaging in the evaluation of patients with Parkinsonism: a US perspective.” Expert Rev. Neurother. 17(3): 219-225.
⁴⁹ Gao, L., et al. (2015). “Cerebrospinal fluid alpha-synuclein as a biomarker for Parkinson's disease diagnosis: a systemic review and meta-analysis.” Int. J. Neurosci. 125(9): 645-654.
⁵⁰ Wick, W., et al. (2012). “Temozolomide chemotherapy alone versus radiotherapy alone for malignant astrocytoma in the elderly: the NOA-08 randomised, phase 3 trial.” Lancet Oncol. 13(7): 707-715.
⁵¹ Clark, C. M., et al. (2003). “Cerebrospinal fluid tau and β-amyloid. How well do these biomarkers reflect autopsy-confirmed dementia diagnoses?” Arch. Neurol. 60(12): 1696-1702.
⁵² Majumder, V., et al. (2018). “TDP-43 as a potential biomarker for amyotrophic lateral sclerosis: a systematic review and meta-analysis.” BMC Neurol. 18: 90.
⁵³ Bendor, J. T., et al. (2013). “The function of α-synuclein.” Neuron 79(6): 1044-1066.
⁵⁴ Spillantini, M. G., et al. (1997). “α-Synuclein in Lewy bodies.” Nature 388(6645): 839-840.
⁵⁵ Fujiwara, H., et al. (2002). “α-Synuclein is phosphorylated in synucleinopathy lesions.” Nat. Cell Biol. 2002; 4(2): 160-164.
⁵⁶ Milanese, C., et al. (2018). “Activation of the DNA damage response in vivo in synocleinopathy models of Parkinson's disease.” Cell Death Dis. 9(8): 818.
⁵⁷ Schaser, A. J., et al. (2019). “Alpha-synuclein is a DNA binding protein that modulates DNA repair with implications for Lewy body disorders.” Sci. Rep. 9(1): 10919.
⁵⁸ Goldstein, M. and Kastan, M. B. (2015). “The DNA damage response: implications for tumor responses to radiation and chemotherapy.” Annu. Rev. Med. 66: 129-143.
⁵⁹ Kam, T., et al. (2018). “Poly(ADP-ribose) drives pathologic α-synuclein neurodegeneration in Parkinson's disease.” Science 362(6414): eaat8407.
⁶⁰ Brown, P. D., et al. (2020). “Hippocampal avoidance during whole-brain radiotherapy plus memantine for patients with brain metastases: phase III trial NRG Oncology CC001.” J. Clin Oncol. 38(10): 1019-1029.
⁶¹ Brown, P. D., et al. (2020). “Hippocampal avoidance during whole-brain radiotherapy plus memantine for patients with brain metastases: phase III trial NRG Oncology CC001.” J. Clin Oncol. 38(10): 1019-1029.
⁶² Mitra, J. and Hegde, M. L. (2019). “A commentary on TDP-43 and DNA damage response in amyotrophic lateral sclerosis.” J. Exp. Neurosci. 13: 1179069519880166.
⁶³ Dormann, D. and Haass, C. (2013). “Fused in sarcoma (FUS): an oncogene goes awry in neurodegeneration.” Mol. Cell Neurosci. 56: 475-486.
⁶⁴ Shoeb, M., et al. (2020). “A possible relationship between telomere length and markers of neurodegeneration in rate brain after welding fume inhalation exposure.” Environ. Res. 180: 108900.
⁶⁵ Pereira C D, et al. (2020). “Nuclear accumulation of LAP1:TRF2 complex during DNA damage response uncovers a novel role for LAP1.” Cells 9(8): 1804.
⁶⁶ Bendor, J. T., et al. (2013). “The function of α-synuclein.” Neuron 79(6): 1044-1066.
⁶⁷ Vasili, E., et al. (2019). “Spreading of α-synuclein and tau: a systematic comparison of the mechanisms involved.” Front. Mol. Neurosci. 12: 107.
⁶⁸ Gibbons, C. H., et al. (2016). “The diagnostic discrimination of cutaneous α-synuclein deposition in Parkinson's disease.” Neurology 87(5): 505-512.
⁶⁹ Isaacson, S. H., et al. (2017). “Clinical utility of DaTscan imaging in the evaluation of patients with Parkinsonism: a US perspective.” Expert Rev. Neurother. 17(3): 219-225.
⁷⁰ Gao, L., et al. (2015). “Cerebrospinal fluid alpha-synuclein as a biomarker for Parkinson's disease diagnosis: a systemic review and meta-analysis.” Int. J. Neurosci. 125(9): 645-654.
⁷¹ Clark, C. M., et al. (2003). “Cerebrospinal fluid tau and β-amyloid. How well do these biomarkers reflect autopsy-confirmed dementia diagnoses?” Arch. Neurol. 60(12): 1696-1702.
⁷² Majumder, V., et al. (2018). “TDP-43 as a potential biomarker for amyotrophic lateral sclerosis: a systematic review and meta-analysis.” BMC Neurol. 18: 90.
⁷³ Wick, W., et al. (2012). “Temozolomide chemotherapy alone versus radiotherapy alone for malignant astrocytoma in the elderly: the NOA-08 randomised, phase 3 trial.” Lancet Oncol. 13(7): 707-715.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

1. A method of preventing or minimizing radiation damage in a subject afflicted with a glioblastoma tumor, comprising:
(i) determining the presence or absence of one or more diagnostic marker of α-synucleinopathy or other markers of central nervous system (CNS) degeneration; and
(ii) treating a subject with one or more diagnostic marker of α-synucleinopathy or other markers of CNS degeneration, with a modified treatment regimen that minimizes the risks associated with the standard Stupp protocol.

2. The method of claim 1, wherein the one or more diagnostic marker of α-synucleinopathy or other markers of CNS degeneration, is selected from the group consisting of:
(i) phosphorylated α-synuclein;
(ii) decreased dopamine levels in the stratum of the subject;
(iii) phosphorylated tau;
(iv) β-amyloid;
(v) transactive response DNA binding protein 43 kDa (TDP-43);
(vi) fused in sarcoma (FUS); and
(vii) telomeric repeat-binding factor 2 (Trf2).

3. The method of claim 2, wherein the phosphorylated α-synuclein is identified in peripheral nerve endings from a skin punch biopsy or as α-synuclein aggregates in a sample of cerebrospinal fluid or blood obtained from the subject.

4. The method of claim 2, wherein the decreased dopamine levels in the stratum of the subject is measured by DaTscan.

5. The method of claim 2, wherein the one or more α-synucleinopathy diagnostic marker is identified in a sample of cerebrospinal fluid or blood obtained from the subject.

6. The method of claim 1, wherein the modified treatment regimen that minimizes the risks associated with the standard Stupp protocol is selected from the group consisting of:
(i) drug therapy alone;
(ii) drug therapy in combination with involved-field radiation administered with a more conformal plan to the tumor while avoiding subcortical regions that are at risk of damage;
(iii) drug therapy in combination with high energy particle radiation administered with a more conformal plan to the tumor while avoiding subcortical regions that are at risk of damage;
(iv) drug therapy in combination with ionizing radiation administered with a more conformal plan to the tumor while avoiding subcortical regions that are at risk of damage;
(v) drug therapy in combination with non-ionizing radiation; and
(vi) non-ionizing radiation alone.

7. The method of claim 6, wherein the drug therapy is selected from the group consisting of: a chemotherapeutic agent, an immunotherapeutic agent, an antibody-drug conjugate, an antiangiogenic agent, a neuroprotection agent, a senolytic agent, and an anti-inflammatory agent.

8. The method of claim 7, wherein the chemotherapeutic agent is selected from the group consisting of: an alkylating agent, an anthracycline, a cytoskeletal disruptor, an epothilone, a histone deacetylase inhibitor, an inhibitor of topoisomerase I, an inhibitor of topoisomerase II, a kinase inhibitor, a nucleotide analog, a peptide antibiotic, a platinum-based agent, a retinoid, and a vinca alkaloid.

9. The method of claim 8, wherein the alkylating agent is selected from the group consisting of: temozolomide, irinotecan, and paclitaxel.
